(19) United States
(12) Patent Application Publication
(10) Pub. No.: US 2003/0036629 A1
(43) Pub. Date: Feb. 20, 2003

(54) NOVEL TGF-BETA PROTEIN PURIFICATION METHODS

(76) Inventors: BARRY FOSTER, CHELMSFORD, MA (US); BONNIE GERMAIN, WEBSTER, NH (US); KAREN HAMMERSTONE, TYNGSBORO, MA (US)

Correspondence Address: FINNEGAN, HENDERSON, FARABOW, GARRETT &, DUNNER LLP, 1300 I STREET, NW, WASHINGTON, DC, 20006, US

(21) Appl. No.: 08/989,543

(22) Filed: Dec. 12, 1997

(51) Int. Cl. C07K 1/00; A61K 38/00; C07K 14/00; C07K 17/00; A23J 1/00; C07K 16/00

(52) U.S. Cl. 530/344; 530/350

(57) ABSTRACT

Methods of purifying TGF-β superfamily proteins, including osteogenic proteins, such as bone morphogenetic proteins (BMPs), are disclosed.

### FIELD OF INVENTION

This present invention relates generally to novel protein recovery and purification methods for the transforming growth factor-β (TGF-β) superfamily of proteins. More particularly, this invention relates to novel methods of purification of such proteins, including bone morphogenetic proteins (BMPs).

### BACKGROUND OF THE INVENTION

The transforming growth factor-β superfamily of proteins, including the BMPs and other osteogenic proteins may be produced in cultures (e.g. yeast, E. coli, and mammalian cells) transformed with an expression vector containing the corresponding DNA. The cloning and expression of the transforming growth factor-β superfamily of proteins, including the bone morphogenetic proteins (also termed osteogenic proteins), have previously been described. See, for example, U.S. Pat. Nos. 4,877,864; 5,108,922; 5,013,649; 5,116,738; 5,106,748; 5,187,076; 5,141,905; 5,688,678; 5,661,007; 5,637,480; 5,639,638; 5,658,882; and 5,635,372. Other compositions which may also be useful include Vgr-2, and any of the growth and differentiation factors (GDFs), including those described in PCT publications WO94/15965; WO94/15949; WO95/01801; WO95/01802; WO94/21681; WO94/15966; and others. Also useful in the present invention may be BIP, disclosed in WO94/01557; and MP52, disclosed in PCT publication WO93/16099. The disclosures of all of the above referenced publications are hereby incorporated by reference.

The use of suitably transformed host cells allows for the recombinant production of high levels of protein. For proteins which are secreted from the host cell, purification of the protein of interest generally involves isolation and purification from the host cell culture medium. Typically, the culture medium contains selected nutrients (e.g., vitamins, amino acids, co-factors, minerals) and can contain additional growth factors/supplements, including insulin and possibly additional exogenous proteins. In addition, the conditioned medium often contains not only the secreted protein of interest, but also significant quantities of additional secreted host cell proteins and other host cell substances (e.g. nucleic acids, membrane vesicles). Thus, even though it is expressed at high levels, the product of interest may represent only a minority of all proteins present in the conditioned medium. Not unexpectedly, proteins secreted by transformed host cells may possess characteristics quite similar to those of the product of interest (e.g. charge, molecular size, amino acid composition), thereby placing significant burden on the process used for purification. Certain purification conditions which are effective in avoiding denaturation of the product of interest are ineffective at distinguishing minor differences between secreted proteins, thereby making it extraordinarily difficult to separate the product of interest from all other host cell proteins present.

In addition to the unwanted secreted host cell proteins described above, conditioned medium may also contain products derived from the heterologously-expressed gene encoding the product of interest. These are not desirable for the final drug substance and include, for example, product forms lacking certain post-translational modifications such as glycosylation, sulfation, gamma carboxylation, or other modifications potentially necessary for biological activity (such as processing of precursor forms). In addition, proteolytically-degraded forms of the product of interest may be present in conditioned medium which also need to be removed during purification, but which very closely resemble the product of interest. Unfortunately, most approaches, such as ion exchange chromatography, hydrophobic interaction chromatography, and size exclusion chromatography do not provide the extent of resolution necessary to distinguish the product of interest from the undesired forms of the product. To take full advantage of minor differences between the desired product and contaminants (e.g. small charge differences, small differences in molecular size), the use of strong denaturants is often required. Such denaturants, however, can lead to loss of biological activity, expression of neo-antigenic sites, and can potentially enhance chemical decomposition of selected post-translational modifications.

Typically, researchers have used combinations of traditional chromatographic techniques to purify desired products. Often, such techniques are insufficient for purification of a product to the level of purity and consistency desired for a human therapeutic product. Researchers have attempted to overcome this difficulty by use of affinity chromatography wherein a protein of interest is bound to an immobilized ligand with which it interacts specifically. Following appropriate washing, the desired product can be eluted by disruption of the ligand-protein interaction, often resulting in a significantly more pure eluate. However, in the instance of separation of a desired product from modified forms present in conditioned medium, single step affinity chromatographic techniques are often ineffective, and must be used in conjunction with other affinity resins and/or traditional separation techniques. Unfortunately, using multiple steps to achieve greater resolution can also result in unacceptably low yields. Even high resolution affinity chromatography steps (e.g., immunoaffinity purification using an immobilized monoclonal antibody) may not afford sufficient resolution of the desired product from other components present in the culture medium due to common sites of interaction. For example, where an epitope which is present on the product of interest, is also present in a proteolytically-degraded form of the product or a precursor form of the desired product, both will compete for the same site.

In addition to separating the product of interest from molecules with similar properties (e.g. modified forms of the expressed gene), it is also important to separate the desired product from components present in conditioned medium with which it specifically interacts. Where the protein of interest is positively charged, it will tend to bind to any negatively charged molecules present thereby making purification of the protein by traditional methods very difficult. For example, certain proteins once expressed and secreted actually “bind-back” to the host cell and remain recalcitrantly associated with the host cell making purification without concommitant denaturation virtually impossible.

Accordingly, there continues to exist a need in the art for protein purification methods that effectively overcome all of these difficulties.

### BRIEF SUMMARY OF THE INVENTION

The methods of the present invention are directed to protein purification comprising the steps of applying cell culture medium to a heparin or heparin-like resin, elution with salt to displace the protein of interest, applying the first eluate to a hydrophobic interaction resin, eluting with decreased ionic strength or with non polar solvents to minimize hydrophobic interactions, and then optionally applying the second eluate to an anion exchange resin, used in the non-adsorptive mode, and used in tandem with a cation exchange resin and eluted with salt. Also, optionally, this third eluate is diafiltrated and/or concentrated, using, e.g., a spiral-wound membrane cartridge or other suitable device.

More specifically, conditioned medium containing cell culture is filtered through a filter and loaded onto a Cellufine Sulfate chromatography column. Suitable heparin or heparin-like resins include those resins having a negatively charged group such as heparin, sulfated esters of cellulose, sulfylpropyl (SP), carboxyl, and carboxy methyl and include Matrex Cellufine Sulfate, Heparin Sepharose, Heparin Toyopearl, Carboxy Sulfon, Fractogel EMD-SO₃, and Fractogel-EMD COO, with the preferred being Matrex Cellufine Sulfate. The column is washed and then eluted to collect, e.g., BMP. A suitable first wash comprises a salt solution such as sodium chloride, potassium chloride, sodium sulphate, sodium phosphate, or potassium phosphate, and optionally, may contain a suitable buffering agent. Suitable concentration ranges are those which are effective in washing without eluting BMP and include for example 5 mM to 600 mM salt, and preferably is 50 mM Tris, 500 mM sodium chloride. The first eluant comprises 50 mM Tris, 0.5 M NaCl, 0.5 M arginine; suitable concentration ranges are those which are effective in eluting BMP, including for example a solution containing a buffering agent at pH about 8.0, such as Tris, in the range of 5 to 100 mM, preferably approximately 50 mM, a salt such as NaCl in the range of 200 to 1000 mM, preferably 500 mM, and arginine in the range of 0 to 1000 mM, preferably 500 mM.

This first eluate is applied to a Butyl Sepharose column which is washed with a suitable second wash which comprises a salt solution such as sodium chloride, ammonium sulfate, potassium chloride, sodium sulphate, sodium phosphate, or potassium phosphate, and optionally, may contain a suitable buffering agent. Suitable concentration ranges are those which are effective in washing the column, without eluting BMP, and include for example 750 mM to 1250 mM salt, and preferably is 50 mM Tris, 1000 mM sodium chloride. The second eluant is one which is sufficient to elute the protein of interest, for example one which comprises 50 mM Tris, 0.5 M arginine, 20% propylene glycol; suitable concentration ranges are those which are effective in eluting BMP, and include for example a solution containing a buffering agent at pH about 7.0, such as Tris, or its equivalent, in the concentration range of 5 to 100 mM, preferably approximately 50 mM, arginine, or its equivalent, in the range of 250 mM to 1000 mM, preferably approximately 500 mM, and a nonpolar solvent, such as propylene glycol, or its equivalent, in the range of 10% to 50%, preferably approximately 20%.

The eluate of the Butyl Sepharose column (referred to herein as the second eluate) is optionally pumped through a DEAE anion exchange resin; the unbound flow-through is pumped into a Carboxy Sulfon cation exchange resin connected in tandem to the DEAE resin. The DEAE and Carboxy Sulfon columns are washed, disconnected, and then the Carboxy Sulfon column is eluted with salt to collect BMP (referred to herein as the third eluate). Suitable anion exchange resins include those resins having a positively charged group such as diethyleaminoethane (DEAE), polyethyleneimine (PEI), and quarternary aminoethane (QAE) and include Q-Sepharose Fast Flow, DEAE-Sepharose Fast Flow, POROS-Q, Fractogel-TMAE, Fractogel-DMAE, QAE-Toyopearl, and DEAE-Toyopearl with the preferred resin being DEAE-Toyopearl (Tosohaas). Suitable cation exchange resins include those having a negatively charged group such as heparin, sulfated esters of cellulose, sulfylpropyl (SP), carboxyl, and carboxy methyl and include Matrex Cellufine Sulfate, SP-Sepharose Fast Flow, Mono S, Resource-S, Source S, Carboxy Sulfon, Fractogel EMD-SO₃, and Fractogel-EMD COO, with the preferred being Carboxy Sulfon. A suitable third wash comprises a salt solution such as sodium chloride, potassium chloride, sodium sulphate, or ammonium sulfate, and may contain a suitable buffering agent and optionally arginine. Suitable concentration ranges are those which are effective in washing without eluting BMP and include for example 0 mM to 250 mM salt, and preferably is 50 mM potassium phosphate, 250 mM arginine. The third eluant comprises 50 mM potassium phosphate, 400 mM NaCl, and 500 mM arginine; suitable concentration ranges are those which are effective in eluting BMP, including for example a solution containing a buffering agent at pH about 7.5, such as potassium phosphate, in the range of 5 to 100 mM, preferably approximately 50 mM, a salt such as NaCl in the range of 200 to 1000 mM, preferably 400 mM or higher, and arginine in the range of 0 to 1000 mM, preferably 500 mM.

Optionally, a spiral-wound membrane cartridge is used to exchange the Carboxy Sulfon elution buffer into a suitable formulation buffer. Immediately after this diafiltration step, the BMP may be concentrated to ≧2.4 absorbance units/mL (at 280 nm) using the spiral-wound cartridge, if necessary. The concentrated BMP is then filtered, sampled, labeled, and stored frozen at −80° C.

The effectiveness of the process in purifying BMP is demonstrated by SDS-PAGE analysis. After the Cellufine Sulfate step, BMP is clearly visible as two major bands in the 15-20 kd region on a reduced gel, although other contaminating proteins are still present. These protein contaminants are largely removed by the Butyl Sepharose and are further separated by the DEAE/Carboxy Sulfon step.

Also provided by the present invention are purified BMP compositions produced by the methods of the invention.

### DETAILED DESCRIPTION OF THE INVENTION

As used herein, the term “BMP” includes, but is not limited to proteins of the transforming growth factor-β superfamily of proteins, including the BMPs, isolated from a variety of tissue sources (including but not limited to epidermis, tendon, bone, cartilage, blood, fetal tissue, neuronal tissue, liver, ligament, muscle, pancreas, lung, heart, spleen, kidney), from transformed cell lines, and recombinantly produced proteins isolated from host cell culture medium or microbial sources (including, but not limited to fermentation broth, E.coli lysate, yeast lysate, and the like).

As used herein, the terms “heparin” resin and “heparin-like” resin are used interchangeably, and include but are not limited to, resins containing an immobilized negatively charged moiety such as heparin, sulfated esters of cellulose, sulfylpropyl (SP), carboxyl, and carboxy methyl and includes Fractogel-EMD-SO₃, Carboxy Sulfon, Fractogel-EMD-COO, Heparin-Sepharose, Matrex Cellufine Sulfate and equivalents thereof, with Matrex Cellufine Sulfate presently most preferred.

As one skilled in the art readily appreciates, the “first wash” can be any salt solution and includes, for example, sodium chloride, potassium chloride, sodium sulphate, sodium phosphate, or potassium phosphate, and can be suitably buffered. Typically, concentrations range from low (5 mM salt) to high (600 mM salt), with 500 mM sodium chloride presently preferred.

As used herein, the term “first eluant” includes, but is not limited to, solutions composed of a buffering agent (e.g. Tris) at a concentration of approximately 50 mM, salt (e.g. NaCl) at a concentration which is sufficient for elution from the resin (e.g. approximately 500 mM), at about pH 8.0, and about 500 mM arginine; suitable concentration ranges are those which are effective in eluting BMP, including for example a solution containing a buffering agent at pH about 8.0, such as Tris, in the range of 5 to 100 mM, preferably approximately 50 mM, a salt such as NaCl in the range of 200 to 1000 mM, preferably 500 mM, and arginine in the range of 0 to 1000 mM, preferably 500 mM.

As used herein, the term “Butyl Sepharose-like” includes, but is not limited to Butyl Sepharose 4B, Butyl Sepharose Fast Flow, Butyl-Toyopearl, and other hydrophobic interaction media including Phenyl Sepharose Fast Flow, Phenyl Toyopearl, Phenyl Fractogel, Butyl Fractogel, and suitable equivalents, with Butyl Sepharose 4B presently being most preferred.

As used herein, the “second wash” can be any salt solution and includes, for example, sodium chloride, potassium chloride, sodium sulphate, ammonium sulfate, sodium phosphate, or potassium phosphate, and can be suitably buffered. Typically, concentrations range from low (750 mM salt) to high (1250 mM salt), with 50 mM Tris, 1000 mM sodium chloride presently preferred.

As used herein, the term “second eluant” includes, but is not limited to, solutions comprising a buffering agent (e.g. Tris) at a concentration of approximately 5 to 100 mM, preferably 50 mM, a solubility-promoting agent (e.g. arginine, urea, or other equivalent chaotropic agent), preferably arginine at a concentration range of approximately 250 mM to 1000 mM, preferably approximately 500 mM, and a nonpolar solvent (e.g. propylene glycol, ethylene glycol, glycerol and equivalents) at a concentration sufficient to disrupt the interaction of BMP with the Butyl Sepharose, at approximately pH 7.0, at a concentration range of approximately 10% to 50% and preferably propylene glycol, or its equivalent, at approximately 20%. As used in this process, the second eluant is preferably compatible with the subsequent process step, including dilution or diafiltration prior to loading into the next step.

As used herein, the term “anion exchange resin” includes, but is not limited to, resins having a positively charged moiety (at neutral pH), such as diethyleaminoethane (DEAE), polyethyleneimine (PEI), and quaternary aminoethane (QAE) and includes, for example, Q-Sepharose Fast Flow (Pharmacia), DEAE-Sepharose Fast Flow, DEAE-Toyopearl, QAE-Toyopearl, POROS-Q, Fractogel-DMAE, Fractogel EMD-TMAE, Matrex Cellufine DEAE, and the like, with DEAE presently preferred.

As used herein, the term “cation exchange resin” includes, but is not limited to, resins having a negatively charged group such as heparin, sulfated esters of cellulose, sulfylpropyl (SP), carboxyl, and carboxy methyl, and include Matrex Cellufine Sulfate, SP-Sepharose Fast Flow, Mono S, Resource-S, Source S, Carboxy Sulfon, Fractogel EMD-SO₃, and Fractogel-EMD COO, with the presently preferred being Carboxy Sulfon.

As used herein, the term “third wash” can be any salt solution and includes, for example, sodium chloride, potassium chloride, sodium sulphate, or ammonium sulfate, and can be suitably buffered (e.g. Tris, phosphate, or sulfate), and optionally can contain arginine. Typically, salt concentrations range from low (0 mM salt) to high (250 mM salt), with 0 mM sodium chloride presently preferred. The presently preferred “third wash” comprises about 50 mM phosphate buffer and about 250 mM arginine.

As used herein, the term “third eluant” includes, but is not limited to, solutions comprising a buffering agent (e.g. Tris, phosphate, or sulfate) at a concentration range of approximately 5 to 100 mM, preferably 50 mM, a solubility-promoting agent (e.g. arginine, urea, or other chaotropic agents), preferably arginine at a concentration range of 0 to 1000 mM, preferably a concentration of approximately 500 mM, and salt (e.g. sodium chloride, potassium chloride) at a concentration sufficient to disrupt interaction of BMP with the resin (e.g. in the range of 200 to 1000 mM, and preferably, approximately 400 mM or higher).

FIG. 1 provides an overview of the process. While the order of the steps set forth is the presently preferred embodiment, it will be appreciated by one skilled in the art that numerous variations and modifications are possible and that such modifications are within the present invention. For example, the order can be re-configured if desired and steps can be omitted.

[image]

Genes encoding recombinant osteogenic proteins may be expressed in mammalian cell lines such as CHO (Chinese Hamster Ovary), COS, BHK, Balb/c 3T3, 293, and similar cell lines known in the art. The mammalian cells may be grown in any suitable medium known in the art. Suitable cell culture media may contain amino acids vitamins, inorganic salts, glucose, sodium pyruvate, thioctic acid, linoleic acid hydrocortisone, putrescine, recombinant insulin, dextran sulfate, and methotrexate. For example, a suitable medium is a DME/F12 (50:50)-based cell culture medium supplemented with hydrocortisone, putrescine, recombinant insulin, dextran sulfate and methotrexate. Other media, such as α-MEM, Dulbecco's MEM, RPMI 1640, may also be suitable, with suitable supplements as may be necessary. (Freshney, R. I., Culture of Animal Cells, A Manual of Basic Technique, Alan R. Liss, Inc., New York (1983)). The cells may be grown in the presence or absence of a serum supplement such as fetal bovine serum (FBS). The cells may be grown in monolayer or suspension culture, and additionally may be grown in large production scale batches. Incorporated by reference are the disclosures of WO 95/12664 (GI 5217-PCT) relating to methods and nutrient media useful for adapting mammalian cell lines to culture densities, and of pending U.S. Ser. No. 08/481,774 (GI 5233) relating to a cell culture medium for production of dimeric proteins.

Any cell capable of producing a protein of the TGF-β superfamily of proteins may be used in the method of the present invention. Transformed CHO cells are the preferred host cells used to produce an osteogenic protein, such as BMPs, particularly BMP-2, in accordance with the present invention. The cell growth medium may be supplemented with FBS to improve the growth of transformed CHO cells in culture. If it is desired to add FBS, concentrations of FBS as low as 0.5% (v/v) may be added. However, addition of animal-origin proteins always presents the risk of harboring viruses and other deleterious agents. The addition of FBS is not necessary for the practice of the present invention. Serum-free media are preferred for use in producing recombinant osteogenic proteins in accordance with the present invention.

CHO cells are known to release lipids, carbohydrates, nucleic acids and C-type (defective retroviral-like) particles into conditioned media. Therefore, the capacity of a purification process to remove and/or inactivate host derived contaminants which may be present is an important aspect of the process. CHO cell protein removal is confirmed by intentionally mixing radiolabeled CHO cell protein with load material and quantifying the reduction at each step. A reduction factor for host cell protein contaminants at each step of purification, and overall, is estimated by introducing concentrations of CHO cell protein which are higher than that expected during normal production.

The C-type particles present in CHO cells have never been demonstrated to be infectious. However, removal or inactivation of these particles during the purification process is still considered desirable. A consensus set of viruses are used to estimate removal/inactivation potential of the purification steps. These viruses have been chosen to represent different size ranges and types (e.g., enveloped/non-enveloped, DNA containing/RNA containing). Included is a murine retrovirus (Murine Xenotropic Leukemia virus) and others that are human pathogens for which CHO cells are permissive (Parafluenza 3 and Retrovirus 3). Simian virus 40 is also included to investigate a more resistant virus. In these studies, virus is introduced into the process at each chromatographic step and the removal/inactivation determined.

Most media components are small chemicals, including salts, amino acids and sugars that do not generally co-purify with the protein of interest over chromatographic columns and are generally not retained by a diafiltration membrane. However, large polymers, such as dextran sulfate and polyvinyl alcohol, which are useful media additives, may specifically interact with the product of interest and often do co-purify. These components must therefore be purified away from the protein of interest. For example, one dextran sulfate useful in the media for producing recombinant proteins such as BMP has a molecular weight of 5,000 and sulfur content 18% (Sigma catalogue #D-7037). Another dextran sulfate has a molecular weight of 500,000 and a sulfur content of 17% (Pharmacia). Incorporated herein by reference is U.S. Pat. No. 5,516,654 (GI 5180A), which relates to a method of protein production wherein dextran sulfate is added to the culture medium. In accordance with the present invention, dextran sulfate may be added to the growth medium at a range of concentrations of from about 1 to about 500 μg/mL, preferably about 200 μg/ML dextran sulfate.

Methotrexate and other selectable markers, which are often used in small volume in the early production of cell cultures, may be toxic. Their removal from the protein preparation is an important step (e.g., the Matrex Cullufine Sulfate Step which provides a 3,540-fold removal) of the purification process (see Table 8).

Expression of an osteogenic protein, such as BMP-2, can be achieved by inserting a suitable gene into an expression vector, inserting this vector into a mammalian cell, and selecting for cells which express the osteogenic protein. For example, vectors encoding BMP-2 are described in U.S. Pat. No. 5,013,649, the contents of which are incorporated herein by reference.

The yield of recombinant osteogenic protein, such as BMP-2, from mammalian cells which express the BMP-2 gene may be measured by known methods such as radioactively labeling cells with [³⁵S] methionine and analyzing secreted proteins by polyacrylamide gel electrophoresis (PAGE) and autoradiography. For measurement of BMP-2 expression from production-scale batches, the amount of functional BMP-2 secreted can be quantitated by bioassay or chromatographic assay methodologies. Any appropriate bioassay may be used, for example, assay of induction of alkaline phosphatase activity in a BMP-2-responsive cell line, or assay of ectopic bone formation in a mammal such as rat, rabbit, cat or dog. Any chromatographic assay method which separates the product of interest form contaminants may be used, including RP-HPLC.

While the examples below describe the present invention being carried out with a cell line which encodes BMP-2, these examples are not limiting. The present invention may also be used with similar results for other protein members of the transforming growth factor beta superfamily, particularly the bone morphogenetic proteins, including BMP-1 through BMP-15. Osteogenic proteins of the BMP family are a promising development in the bone and cartilage field. The BMP family of proteins includes BMPs 1 through 15, and proteins which are encoded by DNA sequences which hybridize thereto under stringent conditions. The following examples illustrate practice of the invention. These examples are for illustrative purposes only and are not intended in any way to limit the scope of the invention claimed. Example 1 describes the heparin/heparin-like hydrophobic affinity step; Example 2 relates to the hydrophobic interaction chromatography step; Example 3 describes the purification steps using tandem anion-cation exchange on Matrex Cellufine Sulfate; Example 4 relates to further purification using the diafiltration/concentration step; and Example 5 describes the purity testing.

### EXAMPLES

### Example 1

### Heparin/Heparin-Like Hydrophobic Affinity Step

Upon secretion from the host cell, the secreted protein is positively charged and binds tightly to the outer surface of the host cell which is negatively charged. A preferred way to disrupt this binding to the outside of the host cell, without destroying the protein of interest and/or without disruption and further leakage of the host cell contents into the medium, is to add dextran sulfate to the culture medium. Although this has the desired effect of disrupting the interaction with the host cell, it creates another problem, namely the binding of the protein of interest to the dextran sulfate which further complicates the purification process.

Surprisingly, it has been found that a heparin or heparin-like resin will effectively compete with with dextran sulfate for binding to BMP so that such resin can be effectively employed to separate the BMP from the dextran sulfate.

Matrex Cellufine Sulfate (Amicon) is used as an affinity matrix for purification of rhBMP-2 from conditioned medium. This resin is composed of spheroidal cellulose beads functionalized with sulfate esters and functions as a heparin analog for purification of heparin-binding proteins. The resin efficiently competes with dextran sulfate present in cell culture medium for binding to rh BMP-2 at pH 8.0. Elution of the bound rhBMP-2 is achieved by using 0.5M L-arginine added to 50 mM TRIS plus 0.5M NaCl.

Cellufine Sulfate, or an equivalent chromatography column, is the first step in the purification of BMP. Conditioned medium is filtered and titrated to pH 8.0±0.2. The titrated material is loaded onto an equilibrated Cellufine Sulfate column at a linear flow rate of ≦3 cm/min. The column is then washed (50 mM TRIS, 0.5M NaCl, pH 8.0) and may be reverse eluted (50 mM TRIS, 0.5M NaCl, 0.5M L-arginine-HCl, pH 8.0). The column eluate is collected as a single eluting peak, approximately one column volume. Suitable operating parameters are described in Table 1.

TABLE 1
Operating Parameters for Cellufine Sulfate Column Step

| Purification Procedures | Parameter | Target Range | |
|---|---|---|---|
| All Procedures | Pressure | ≦20 | psig |
| Equilibration | Flow Rate | ≦180 | cm/hr |
| | pH | 8.0 ± 0.2 | |
| | Conductivity | ≦33.5 | mS/cm |
| Titration | Volume | 50-125 | mL titrant/L cell |
| | | | culture medium |
| | pH | 8.0 ± 0.2 | |
| | Conductivity | 5-20 | mS/cm |
| Load | Flow Rate | ≦180 | cm/hr |
| Wash | Volume | ≦15 | column volumes |
| Elution | Flow Rate | ≦180 | cm/hr |

### Example 2

### Hydrophobic Interaction Chromatography Step

The heparin-like step effectively removes various species of protein contaminants, methotrexate, dextran sulfate and DNA. Still present in the first eluate, along with the protein of interest, are various forms of the BMP at various stages of proteolytic processing, including the higher molecular weight precursor forms (approximately 110 KD and 80 KD on non-reducing SDS-PAGE analysis) and the desired product (15 KD-20 KD on reducing SDS-PAGE analysis). Because these various species differ only slightly in hydrophobicity, it is difficult to purify these species from each other by conventional methods. Surprisingly, it has been found that the Butyl Sepharose allows for separation of the various forms of BMP-2 by an unconventional use of displacement chromatography. The precursor species of BMP-2 compete with the desired product for binding to the resin. Due to the slight differences in hydrophobicity, the desired product, which is more hydrophobic, binds more tightly to the resin; this allows the other species to be removed in the column load and wash.

Butyl Sepharose 4B (Pharmacia) is a resin used for purification of BMP in a hydrophobic interaction chromatography (HIC) mode. This resin is composed of butylamine coupled to CNBr-activated Sepharose 4B. Butyl Sepharose, or an equivalent column, is the second step of the present invention. Proteins are bound to HIC resins at high conductivities, which promote hydrophobic interactions. “High conductivity” is a minimum value of about 50 mS/cm. Elution is accomplished by decreasing ionic strength and/or by addition of non-polar solvents to minimize hydrophobic interactions. Decreased ionic strength is defined as decreasing the conductivity to a value of below about 20 mS/cm. Non-polar solvents include propylene glycol, ethylene glycol, glycerol, and equivalents thereof.

The Matrex Cellufine Sulfate elution peak is adjusted to pH 7.0±0.2 and 1000 mM NaCl with 200 mM MES, 4000 mM NaCl, 500 mM L-arginine HCl, pH 6.8. MES is 2-[N-morpholino]ethane sulfonic acid. This material is then loaded onto an equilibrated Butyl Sepharose, or an equivalent column, at a flow rate of ≦30 cm/hr. The column is then washed (50 mM TRIS, 1000 mM NaCl, 500 mM L-arginine-HCl, pH 7.0) and bound rhBMP-2 is optionally reverse eluted with 50 mM TRIS, 20% propylene glycol, 500 mM L-arginine-HCl, pH 7.0. The column eluate is collected as a single elution peak, approximately 1.5 column volumes. Suitable column operating parameters are detailed in Table 2.

TABLE 2
Operating Parameters for Butyl Sepharose Column Step

| Purification Procedures | Parameter | Target Range | |
|---|---|---|---|
| All Procedures | Pressure | ≦7 | psig |
| Equilibration | Flow Rate | ≦30 | cm/hr |
| | pH | 7.0 ± 0.2 | |
| | Conductivity | 50-70 | mS/cm |
| Titration | pH | 7.0 ± 0.2 | |
| | Conductivity | 60-70 | mS/cm |
| Load | Flow Rate | ≦30 | cm/hr |
| Wash | Flow Rate | ≦30 | cm/hr |
| | Volume | 2.5-2.75 | column volumes |
| Elution | Flow Rate | ≦15 | cm/hr |

### Example 3

### Tandem Anion-Cation Exchange

The Butyl Sepharose step shows removal of CHO protein contaminants, DNA, and BMP related species, other than the defined product. It has been found that inclusion of an anion exchange chromatography step results in increased removal of DNA, and other non-proteinaceous contaminants. An additional cation exchange chromatography step provides further removal of CHO protein contaminants and concentration of BMP.

Toyopearl-DEAE (TosoHaas) is a weak anion exchange resin that binds negatively-charged proteins and other contaminants on the basis of their charge. It is used in the nonadsorptive mode for purification of BMP, such that it does not bind to the resin, but negatively charged contaminants are able to bind to the DEAE resin.

Carboxy Sulfon (J. T. Baker, Inc.) is a silica-based matrix functionalized with mixed weak and strong cation exchange groups (i.e., carboxy and sulfone groups). BMP binds to Carboxy Sulfon via charge interactions and is eluted by disruption of these interactions using buffers with increased ionic strength.

The final chromatographic step in the purification of BMP is composed of these two ion-exchange columns, or their equivalents, operated in tandem: Toyopearl-DEAE anion exchange column (or its equivalent) followed by Carboxy Sulfon cation exchange column (or its equivalent). The inlet to the DEAE/Carboxy Sulfon system enters the DEAE column first. The outlet of the DEAE column is then plumbed to the inlet of the Carboxy Sulfon column.

The Butyl Sepharose peak is diluted with 9-11 volumes of (50 mM potassium phosphate, 0.25M L-arginine-HCl, pH 7.6). This solution is pumped through the DEAE column and onto the Carboxy Sulfon column at a flow rate of ≦300 cm/hr. The columns are then washed (50 mM potassium phosphate, 0.25M L-arginine-HCl, pH 7.6). The two columns are disconnected and the BMP bound to the Carboxy Sulfon column is eluted with 50 mM potassium phosphate, 0.5M L-arginine-HCl, 0.4M NaCl, pH 7.5. The column eluate is collected as a single eluting peak, approximately 1 column volume. Suitable operating parameters for these columns are detailed in Table 3.

TABLE 3
Operating Parameters for DEAE/Carboxy Sulfon Column Step

| Purification Procedures | Parameter | Target Range | |
|---|---|---|---|
| All Procedures | Pressure | ≦20 | psig |
| Charge Resin | Flow Rate | ≦300 | cm/hr |
| | Volume | ≧1 | column volume |
| Equilibration | Flow Rate | ≦300 | cm/hr |
| | LAL | <4 | EU/mL |
| | pH | 7.6 ± 0.2 | |
| | Conductivity | 10-18 | mS/cm |
| Dilution | pH | 7.6 ± 0.2 | |
| | Conductivity | 10-18 | mS/cm |
| Load | Flow Rate | ≦300 | cm/hr |
| Wash | Flow Rate | ≦300 | cm/hr |
| | Volume | ≧6 | column volumes |
| Elution | Flow Rate | ≦120 | cm/hr |

### Example 4

### Diafiltration/Concentration Step

This is an optional “finishing” step. Tangential flow filtration is used for buffer exchange and concentration of protein solutions. Membrane of specified molecular weight cut-offs are used to retain large molecular weight components (e.g., BMPs) while lower molecular weight components (e.g., salts) are removed. By continuously adding new buffer to the retentate, at a similar rate that solution is passing through the filter, the original buffer components will gradually be diluted away. In this continuous diafiltration fashion, replacement of 5 retentate volumes of a new buffer will effectively replace ≧98% of the original buffer. Without addition of new buffer, a protein solution is concentrated without altering the buffer composition.

The final step in the purification process involves exchange of the Carboxy Sulfon elution buffer into an appropriate formulation buffer for BMP. This is followed by concentration of the material to ≧2.4 absorbance units (at 280 nm), as necessary. This step may be performed using a spiral-wound 10,000 MW cut-off membrane, or equivalent. The Carboxy sulfon eluate is placed in a clean, autoclaved and sealed vessel. The material in the vessel is then pumped across the membrane, at a positive transmembrane pressure, and recirculated back into the vessel. The positive transmembrane pressure forces low-molecular weight solutes through the membrane. Buffer solution (0.01M L-histidine, 0.5M L-arginine-HCl or other suitable buffer solution) enters the vessel at approximately the same rate as material flows through the membrane, thereby diluting out the Carboxy Sulfon elution buffer. This diafiltration process is continued until at least 5 volumes of buffer solution have flowed into the vessel.

After the diafiltration is complete, the valve that allows buffer solution to enter the vessel is closed. To concentrate BMP, the system pump is restarted and material is filtered until a concentration of ≧2.4 absorbance units (at 280 nm) is obtained. The BMP buffer is pumped out of the vessel, through a 0.2 μm filter, and into appropriately sized Teflon bottles. The material is sampled, weighed, labeled, and stored at −80° C. Suitable operating parameters for this process step are detailed in Table 4.

TABLE 4
Operating Parameters for Diafiltration/Concentration Step

| Purification | | | |
|---|---|---|---|
| Procedures | Parameter | Target Range | |
| All Procedures | Inlet Pressure | 20-30 | psig |
| | Permeate Flow Rate | 30-70 | mL/min |
| Equilibration | Retentate Flow Rate | 700-1000 | mL/min |
| | LAL | <4 | EU/mL |
| | pH | 6.5 ± 0.2 | |
| | Conductivity | 20-30 | mS/cm |
| Diafiltration | Permeate pH | 6.5 ± 0.2 | |
| | Permeate Conductivity | 20-30 | mS/cm |
| | Permeate Volume | ≧5 | times load volume |

### Example 5

### Purity Testing

Studies investigating the effectiveness of the above purification process for removing DNA, viruses, dextran sulfate and methotrexate were performed with the results described in the tables below:

TABLE 5
DNA Removal Studies

| Purification Process Step | Fold Removal | Log Removal |
|---|---|---|
| Matrex Cellufine Sulfate | 251 | 2.40 |
| Butyl Sepharose | 1122 | 3.05 |
| DEAE/Carboxy Sulfon | 15 | 1.18 |
| Overall | | 6.63 |

TABLE 6
MuLV Virus Removal/Inactivation Studies

| Purification Process Step | Fold Removal | Log Removal |
|---|---|---|
| Matrex Cellufine Sulfate | 4677 | 3.67 |
| Butyl Sepharose | 275 | 2.44 |
| DEAE/Carboxy Sulfon | 1950 | 3.29 |
| Overall | — | 9.4 |

TABLE 7
Dextran Sulfate Removal Studies

| Purification Process Step | Fold Removal | Log Removal |
|---|---|---|
| Matrex Cellufine Sulfate | 1318 | 3.12 |
| Butyl Sepharose | 141 | 2.15 |
| DEAE/Carboxy Sulfon | 67.6 | 1.83 |
| Overall | — | 7.10 |

TABLE 8
Methotrexate Removal Studies

| Purification Process Step | Fold Removal | Log Removal |
|---|---|---|
| Matrex Cellufine Sulfate | 3540 | 4.55 |

While the above examples are not limiting, it can be seen that the above process results in a high-fold removal of potential contaminants, including DNA, virus, dextran sulfate and methotrexate, from recombinant osteogenic protein produced from transfected CHO cells.

While the present method of the invention is exemplified by purification of recombinantly produced BMP from transformed host cells, the method is also amenable to purification of BMP naturally occurring within a cell and can be used to purify proteins from solution or from various tissue types, cell homogenates, cell culture supernatants, or isolated cellular sub-fractions. While the present invention has been described in terms of specific methods and compositions, it is understood that variations and modifications will occur to those skilled in the art upon consideration of the present invention.

Numerous modifications and variations in the invention as described in the above illustrative examples are expected to occur to those skilled in the art and, consequently, only such limitations as appear in the appended claims should be placed thereon. Accordingly, it is intended in the appended claims to cover all such equivalent variations which come within the scope of the invention as claimed.

What is claimed:

1. A method for purification of a TGF-β superfamily protein in a solution comprising the steps of:
applying said solution to a heparin-like resin,
eluting said heparin-like resin with a first eluant to form a first eluate,
applying said first eluate to a Butyl Sepharose-like resin,
eluting said Butyl Sepharose-like resin with a second eluant to form a second eluate containing said TGF-β superfamily protein.

2. The method of claim 1, further comprising the steps of:
applying said second eluate to an ion exchange resin, and
eluting said ion exchange resin with a third eluant to form a third eluate.

3. The method of claim 2, wherein said ion exchange resin is a resin selected from the group consisting of an anion exchange resin and a cation exchange resin.

4. The method of claim 1, wherein said heparin-like resin has a negatively charged group which is a member selected from the group consisting of heparin, sulphated esters of cellulose, sulfylpropyl (SP), carboxyl, and carboxy methyl.

5. The method of claim 4 wherein said heparin-like resin is Matrex Cellufine Sulfate.

6. The method of claim 1, wherein said first eluant comprises a salt.

7. The method of claim 6, wherein said first eluant comprises 50 mM Tris, 0.5 M NaCl, 0.5 M L-arginine.

8. The method of claim 1, wherein said Butyl Sepharose-like resin is a member selected from the group consisting of Butyl Sepharose 4B, Butyl Sepharose Fast Flow, and Butyl-Toyopearl.

9. The method of claim 8, wherein said Butyl Sepharose-like resin is Butyl Sepharose 4B.

10. The method of claim 1, wherein said second eluant comprises a buffering agent, a chaotropic agent, and a non-polar solvent.

11. The method of claim 10, wherein said second eluant is about 50 mM Tris, 500 mM arginine, and 20% propylene glycol.

12. The method of claim 3, wherein said anion exchange resin has a positively charged group which is a member selected from the group consisting of: diethyleaminoethane (DEAE), polyethyleneimine (PEI), and quaternary aminoethane (QAE).

13. The method of claim 12, wherein said anion exchange resin is DEAE.

14. The method of claim 3, wherein said cation exchange has a negatively charged group which is a member selected from the group consisting of as heparin, sulfated esters of cellulose, sulfylpropyl (SP), carboxyl, and carboxy methyl.

15. The method of claim 14, wherein said cation exchange resin is Carboxy Sulfon.

16. The method of claim 1, wherein said third eluant comprises a buffering agent, a solubility-promoting agent, and a salt.

17. The method of claim 16, wherein said third eluant comprises about 50 mM Tris, 500 mM arginine, and 400 mM sodium chloride.

18. The method of claim 1, wherein said TGF-β superfamily protein is a BMP.

19. The method of claim 18, wherein said BMP is BMP-2.

20. A BMP produced by the method of claim 18.

21. A method for purification of BMP-2 in a solution comprising the steps of:
applying said solution to a Cellufine Sulfate resin,
eluting said Cellufine Sulfate resin with a first eluant to form a first eluate,
applying said first eluate to a Butyl Sepharose 4B resin,
eluting said Butyl Sepharose 4B resin with a second eluant to form a second eluate, containing said BMP-2.

22. The method of claim 21 further comprising the steps of:
applying said second eluate containing said BMP-2, to a DEAE resin,
washing said DEAE resin to form a third wash,
applying said wash to a Carboxy Sulfon resin, and
eluting said Carboxy Sulfon resin with a third eluant to form a third eluate containing said BMP-2.

23. The method of claim 22, wherein:
said first eluant comprises about 50 mM Tris, 500 mM NaCl, and 500 mM arginine;
said second eluant comprises about 50 mM Tris, 500 mM arginine, and 20% propylene glycol;
said third wash comprises about 50 mM potassium phosphate and 250 mM arginine; and
said third eluant comprises about 50 mM Tris, 500 mM arginine, and 400 mM sodium chloride.
